(19) United States
(12) Patent Application Publication
(10) Pub. No.: US 2019/0204008 A1
(43) Pub. Date: Jul. 4, 2019

(54) ABOVE CRYOGENIC SEPARATION PROCESS FOR PROPANE DEHYDROGENATION REACTOR EFFLUENT

(71) Applicant: SABIC Global Technologies B.V., Bergen op Zoom (NL)

(72) Inventor: Joris VAN WILLIGENBURG, Geleen (NL)

(21) Appl. No.: 16/328,053

(22) PCT Filed: Aug. 21, 2017
(86) PCT No.: PCT/IB2017/055040

§ 371 Date: Feb. 25, 2019

Related U.S. Application Data
(60) Provisional application No. 62/379,476, filed on Aug. 25, 2016.

(51) Int. Cl. F25J 3/02; C07C 7/09; C07C 7/04; C07C 7/00; C07C 5/333

(52) U.S. Cl. CPC F25J 3/0219; F25J 2220/68; F25J 3/0242; F25J 3/0252; C07C 7/09; C07C 7/04; C07C 7/005; C07C 5/333; F25J 2200/02; F25J 2200/74; F25J 2205/04; F25J 2210/12; F25J 2215/64; F25J 2270/06; F25J 2270/60; F25J 2220/66; F25J 3/0233

(57) ABSTRACT

Systems and methods for separating effluent from a propane dehydrogenation reactor to recover propylene are disclosed. The systems and methods involve using turbo-expanders in a cooling process that does not cool below −140° C. and may also use a de-ethanizer unit to remove ethane and components more volatile than ethane from propylene streams.

### CROSS REFERENCE TO RELATED APPLICATIONS

This application claims the benefit of priority of U.S. Provisional Patent Application No. 62/379,476, filed Aug. 25, 2016, which is hereby incorporated by reference in its entirety.

### A. FIELD OF INVENTION

The present invention relates to separation processes for recovering propylene and/or hydrogen from propane dehydrogenation reactor effluents.

### B. DESCRIPTION OF RELATED ART

Propylene is an unsaturated hydrocarbon that is a key petroleum building block of a wide variety of polymers and intermediaries. One process for producing propylene involves dehydrogenating propane. Dehydrogenation, as the name suggests, involves the removal of hydrogen atoms from a compound. In the dehydrogenation of propane, hydrogen is removed from propane to form propylene according to the following reaction:

C₃H₈→C₃H₆+H₂

The dehydrogenation reaction of propane to form propylene is typically carried out in the presence of catalyst in dehydrogenation reactors. The effluent from the dehydrogenation reactors include primarily propylene (C₃H₆, the main product), propane (C₃H₈, unreacted portion of the propane feed that may be recycled back to the dehydrogenation reactor in a further attempt to dehydrogenate it to produce propylene), and hydrogen (H₂, the main byproduct resulting from the dehydrogenation reaction). Both propylene and hydrogen are valuable components of the dehydrogenation reactor effluent.

Side reactions occurring alongside the main dehydrogenation reaction shown above may cause the formation of other hydrocarbons. These other hydrocarbons that may be comprised in the dehydrogenation reactor effluent may be less valuable than propylene and hydrogen, or they may be at such low concentrations that it may not be desirable to recover them separately, or both. Examples of these other materials include methane, ethane, and ethylene, which, collectively, can be used as fuel for the dehydrogenation reactor. The dehydrogenation reactor effluent may also include water and carbon dioxide.

Separating propylene from the other components of the dehydrogenation reactor effluent typically involves cooling the effluent in a heat exchanger and distilling the cooled effluent in a distillation column. However, due to the relatively high hydrogen content in the dehydrogenation reactor effluent, a significant amount of propylene will remain in the gas fraction from the distillation column and thus the propylene in that gas fraction will not be recovered in a pure or substantially pure form. The prophetic simulated example (Example 1) discussed below illustrates this. Example 1 shows that the lower the temperature to which the reactor effluent stream is cooled, the higher the yield of propylene. Thus, propylene is often recovered by cooling the propane dehydrogenation reactor effluent stream to cryogenic temperatures (i.e. a temperature of −100° C. or below). A challenge in propylene recovery is efficiently cooling the reactor effluent to a sufficiently low temperature to obtain high propylene recovery.

Referring to FIG. 3, prior art system 30 for recovering propane and propylene (collectively 99% or more of the reactor effluent) is illustrated. System 30 cools reactor effluent with a series of heat exchangers, to approximately −100° C. (the actual temperature to which the reactor effluent is cooled depends, at least in part, on its composition). The cooling is provided by a propylene compressor refrigeration cycle and an ethylene refrigeration cycle. The ethylene refrigeration cycle discharges heat to the propylene refrigeration cycle.

Reactor effluent gas 3001, which may have been previously compressed, is cooled in heat exchanger H-1 to approximately −35 to −40° C. to form cooled effluent 3002. Cooled effluent 3002 is then cooled to approximately −100° C. in heat exchanger H-2 to form stream 3003. Stream 3003 can then be separated into a stream that primarily comprises propylene and a stream that primarily comprises hydrogen. Heat exchanger H-1 uses propylene refrigerant 3007. Propylene refrigerant 3007 is vaporized by heat exchanger H-1, producing vapor 3008. Vapor 3008 combines with vaporized propylene 3016 to form stream 3004, which is recompressed by compressor K-1 to form stream 3005 under conditions such that stream 3005 can be condensed by cooling water in heat exchanger H-3 to form liquid pressurized propylene 3006. Liquid pressurized propylene 3006 splits to form liquid pressurized propylene 3006A and liquid pressurized propylene 3006B. Liquid pressurized propylene 3006A is flowed through valve V-1, which causes a decrease in pressure over liquid pressurized propylene 3006A. As a result, a portion of liquid pressurized propylene 3006A vaporizes and cools the remainder of liquid pressurized propylene 3006A. The cooled remainder portion of liquid pressurized propylene 3006A forms propylene refrigerant 3007 for heat exchanger H-1. H-1 can be a series of heat exchangers taking propylene refrigerant at multiple temperature/pressure levels. K-1 is typically a multi stage compressor, taking propylene vapors at different pressure levels and providing propylene refrigerant at different pressure levels to H-1.

Heat exchanger H-2 uses ethylene refrigerant. Heat exchanger H-2 vaporizes ethylene refrigerant 3014 to form vaporized ethylene refrigerant 3015. Vaporized ethylene refrigerant 3015 is at low pressure and is used by heat exchanger H-4 to cool high-pressure ethylene vapor 3012 from heat exchanger H-3. Ethylene stream 3010 from the heat exchanger H-4 is compressed by compressor K-2, to form ethylene stream 3011 at an elevated temperature. Heat exchanger H-3 cools ethylene stream 3011 to produce high-pressure ethylene 3012, which is cooled in heat exchanger H-4 to cold, high-pressure ethylene 3013.

The cold high-pressure ethylene 3013 is subsequently condensed in heat exchanger H-5. Condensed cold high-pressure ethylene 3013 is flowed through valve V-2, thereby lowering its pressure, after which it is used as ethylene refrigerant 3014 at heat exchanger H-2. The condensation heat in heat exchanger H-5 is removed by vaporing propylene 3009 (after it passed through valve V-3). From heat exchanger H-5, vaporized propylene 3009 combines with vapor 3008 to form stream 3004, which flows to compressor K-1 where it is recompressed. H-2 can be a series of heat exchangers taking liquid refrigerant at different pressure levels and compressor K-2 can be several compressor stages taking ethylene vapor at different pressure levels.

Propylene demand is expected to grow. There exists a need for alternative methods of propylene production and/or recovery that requires low capital investment, is energy efficient, and has relatively low production costs.

### BRIEF SUMMARY OF THE INVENTION

A discovery has been made that provides a process for separating effluent of a propane dehydrogenation reactor to recover propylene and/or hydrogen. The process involves using turbo-expanders in a cooling process that does not cool below −140° C. The process may also include using a de-ethanizer unit to achieve high levels of recovery of propylene.

Embodiments of the invention include a separation process to recover propylene from effluent of a propane dehydrogenation reactor. The process may include cooling the effluent to produce a gas stream, in which hydrogen and propylene collectively comprises the major component of the gas stream. The process may further include cooling the gas stream in a cooling unit that comprises one or more turbo-expanders. The one or more turbo-expanders do not cool any portion of the gas stream below −140° C. The process may further include flowing condensate from the cooling unit to a de-ethanizer unit, where the de-ethanizer unit is adapted to remove ethane and components just as volatile as or more volatile than ethane under conditions in the de-ethanizer unit. The process may further include flowing, from the de-ethanizer unit, a liquid stream comprising propylene.

Embodiments of the invention include a separation process to recover propylene from effluent of a propane dehydrogenation reactor. The process may include cooling of the effluent to produce a gas stream, in which hydrogen and propylene collectively comprises the major component of the gas stream. The cooling of the effluent to produce the gas stream may include heat transfer and separation processes carried out in a series of units, where each unit comprises a heat exchanger that cools influent of the heat exchanger and a vessel that separates effluent of the heat exchanger into vapor and condensate. The process may further include cooling the gas stream in a cooling unit that comprises a cold box, a separation vessel, and one or more turbo-expanders. The cold box may cool the gas stream so that a portion of the gas stream condenses, thereby forming a stream including cold box condensate and cold box vapor. The process may then further include flowing the stream including cold box condensate and cold box vapor to the separation vessel and separating, by the separation vessel, the stream including cold box condensate and cold box vapor into a separate stream of cold box condensate and a separate stream of cold box vapor. The process may further include expanding the separate stream of cold box vapor in the one or more turbo-expanders and flowing the expanded cold box vapor from the one or more turbo-expanders to the cold box to cool the cold box and thereby produce a reheated cold box stream. The process may further include expanding the reheated cold box stream in the one or more turbo-expanders to a temperature of −140° C. or above and flowing the separate stream of cold box condensate to a de-ethanizer, where the de-ethanizer unit is adapted to remove, with a distillation column, ethane and components just as volatile as or more volatile than ethane under conditions in the distillation column. From the distillation column, a liquid stream is flowed comprising more than 98% by weight of propylene present in the effluent of the propane dehydrogenation reactor.

The following includes definitions of various terms and phrases used throughout this specification.

The phrase “cryogenic temperature” is a temperature of −100° C. or below.

The phrase “polymer grade propylene” is a product having at least 97 to 99 wt. % propylene.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding of the present invention, reference is now made to the following descriptions taken in conjunction with the accompanying drawing, in which:

FIG. 1 shows a system for separating propylene from a propane dehydrogenation reactor effluent, according to embodiments of the invention;

FIG. 2 shows a diagram of a prophetic simulation example to illustrate the problem in separating hydrogen from propylene;

FIG. 3 shows a prior art system for separating effluent of a propane dehydrogenation reactor;

FIG. 4 shows a prior art system for purifying a C₃ stream; and

FIG. 5 shows a prior art system for purifying a C₃ stream.

### DETAILED DESCRIPTION OF THE DISCLOSURE

A discovery has been made of a process for separating reactor effluent of a propane dehydrogenation reactor, where the reactor effluent comprises propane and propylene (as primary components), and hydrogen. The reactor effluent may also comprise ethane, methane, and other hydrocarbons. The process may separate the reactor effluent into a hydrogen rich stream (e.g., >90% vol. hydrogen), a C₁-C₂ hydrocarbon fraction, a polymer grade propylene fraction, a propane fraction, a C₄₊ fraction or combinations thereof. The process may include flash separation and distillation. The cooling in the process may be provided by a propane compressor refrigeration cycle and/or a propylene compressor refrigeration cycle and a turbo-expander-compressor. In embodiments of the invention, temperatures for the separated fractions may be at or above −140° C., or within the range −140° C. to −135° C., or −135° C. to −130° C. or −130° C. to −125° C., or −125° C. to −120° C., and all ranges and values there between including −139° C., −138° C., −137° C., −136° C., −135° C., −134° C., −133° C., −132° C., −131° C., −130° C., −129° C., −128° C., −127° C., −126° C., −125° C., −124° C., −123° C., −122° C., or −121° C. In embodiments of the invention, temperatures for the separated fractions may remain at or above −120° C., or within the range −120° C. to −115° C., or −115° C. to −110° C. or −110° C. to −105° C., or −105° C. to −100° C., and all ranges and values there between including −119° C., −118° C., −117° C., −116° C., −115° C., −114° C., −113° C., −112° C., −111° C., −110° C., −109° C., −108° C., −107° C., −106° C., −105° C., −104° C., −103° C., −102° C., or −101° C. In embodiments of the invention, temperatures for the separated fractions may remain at or above −100° C.

In embodiments of the invention, cooling of a propane dehydrogenation reactor effluent stream includes cooling that stream in a plurality of heat exchangers arranged in series. The cooled stream from each of the heat exchangers is flowed to a separation vessel for separating vapor from condensate formed by the cooling process. The vapor from each of the separation vessels becomes the feed for the next heat exchanger. In this way, as the less volatile hydrocarbons are condensed and removed as condensate, the vapor stream becomes increasingly concentrated with hydrogen (and other light hydrocarbons) to create a hydrogen rich stream.

In embodiments of the invention, this hydrogen rich stream (from the last separation vessel) may be flowed, for further cooling, to a cooling system having one or more turbo-expanders and a cold box. These various stages of cooling, in embodiments of the invention, do not cool any of the streams below −140° C. and may result in recovery of over 90 wt. % of propylene in a liquid stream and recovery of 90% vol. or more of hydrogen as a byproduct in a vapor stream. Embodiments of the invention may also include the use of a de-ethanizer that receives the condensate from the series of separation vessels to achieve propylene recovery of more than 97% or more by weight of propylene present in the effluent of the propane dehydrogenation reactor.

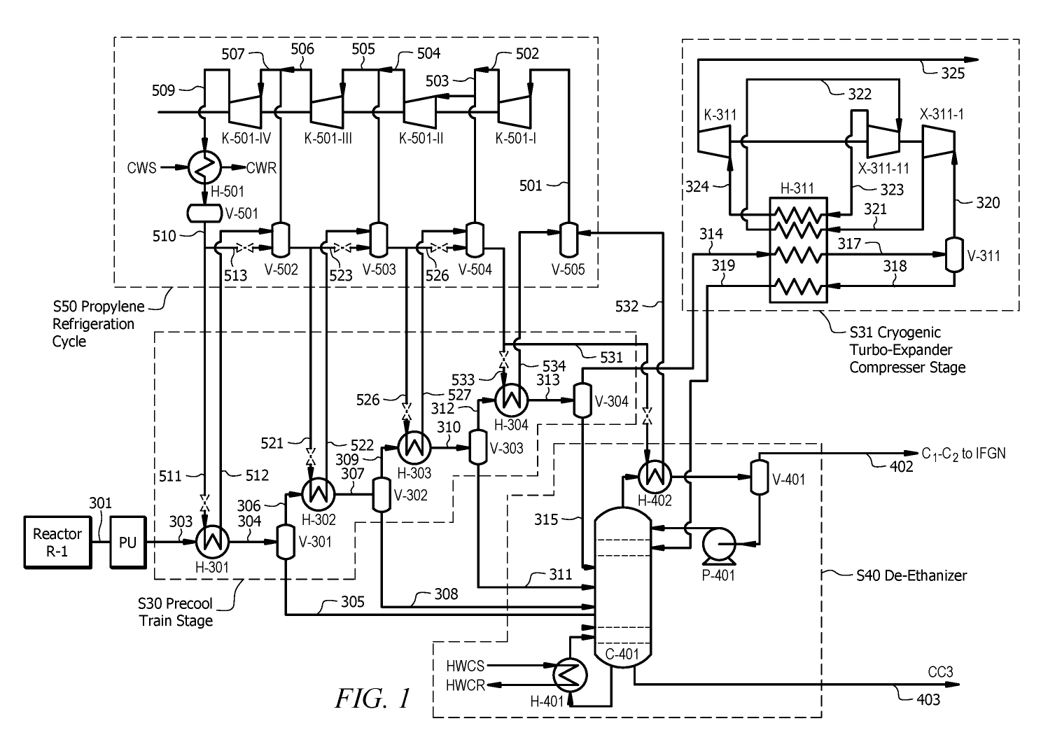

FIG. 1 shows system 10 for separating and recovering the components of a propane dehydrogenation reactor effluent, according to embodiments of the invention. System 10 embodies four major stages of the separation and recovery process, namely, precool train stage S30, cryogenic turbo-expander-compressor separation stage S31, de-ethanizer stage S40, and propylene refrigeration cycle stage S50. In embodiments of the invention, prior to stage S30, reactor effluent pretreatment unit PU may compress reactor effluent gas stream 301 as well as remove carbon dioxide (CO₂) and water from reactor effluent gas stream 301 to form treated effluent gas stream 303. Treated effluent gas stream 303 is flowed to precool train stage S30.

At precool train stage S30, heat exchange equipment cools and partially condenses treated effluent gas stream 303. The heat exchange equipment may cool treated effluent gas stream 303 to a temperature of approximately −35° C., or within a range −45° C. to −25° C. and all ranges and values there between including −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., or −25° C. The heat exchange equipment that implements precool train stage S30 may include one or more heat exchangers and one or more separation vessels. For example, as illustrated in FIG. 1, precool train stage S30 may be implemented by equipment that includes heat exchangers H-301, H-302, H-303, and H-304, arranged in series, for cooling treated effluent gas stream 303. Precool train stage S30 may also involve vessels V-301, V-302, V-303, and V-304 receiving cooled heat exchanger effluents 304, 307, 310 and 313, respectively, from heat exchangers H-301, H-302, H-303, and H-304, respectively. In this way, in embodiments of the invention, treated effluent gas stream 303 may comprise 1 to 7 wt. % hydrogen and ranges and values there between including 1 wt. %, 2 wt. %, 3 wt. %, 4 wt. %, 5 wt. %, 6 wt. %, or 7 wt. % hydrogen and, as the less volatile hydrocarbons are condensed and removed as condensate, the concentration of hydrogen progressively increases such that separator gas stream 314 may comprise 20 to 28 wt. % of hydrogen and ranges and values there between including 20 wt. %, 21 wt. %, 22 wt. %, 23 wt. %, 24 wt. %, 25 wt. %, 26 wt. %, 27 wt. %, or 28 wt. % hydrogen.

Vessels V-301, V-302, V-303, and V-304 separate cooled heat exchanger effluents 304, 307, 310 and 313 into separator gas streams and separator liquid streams. For example, V-301 produces separator liquid stream 305 and separator gas stream 306, V-302 produces separator liquid stream 308 and separator gas stream 309, V-303 produces separator liquid stream 311 and separator gas stream 312, and V-304 produces separator liquid stream 315 and separator gas stream 314.

Separator liquid streams 305, 308, 311, and 315 are routed to de-ethanizer distillation column of de-ethanizer stage S40. Separator liquid streams 305, 308, 311, and 315 may include primarily propylene and propane (propylene typically being the larger component). For example, in embodiments of the invention, separator liquid streams 305, 308, 311, and 315 may comprise propylene in the range 45 wt. % to 60 wt. % and ranges and values there between including 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, or 60 wt. % And separator liquid streams 305, 308, 311, and 315 may comprise propane in the range 40% to 45 wt. %, and ranges and values there between including 40 wt. %, 41 wt. %, 42 wt. %, 43 wt. %, 44 wt. %, or 45 wt. %

Separator gas streams 306, 309, and 312 are each cooled by heat exchangers H-302, H-303, and H-304 to form heat exchanger effluents 307, 310, and 313, respectively. Each of heat exchanger effluents 307, 310 and 313 has a condensed liquid portion and a gas portion. And each of heat exchanger effluents 307, 310 and 313 is flowed to the next separation vessel (vessels V-302, V-303, and V-304, respectively). From the last vessel in the series, vessel V-304, separator gas stream 314 flows to cold box H-311 of cryogenic turbo-expander-compressor separation stage S31.

In embodiments of the invention, cryogenic turbo-expander-compressor separation stage S31 may cool separator gas stream 314. Separator gas stream 314 typically includes primarily hydrogen and propylene. In embodiments of the invention, separator gas stream 314 may comprise propylene in the range 24 to 32 wt. % and ranges and values there between including 24 wt. %, 25 wt. %, 26 wt. %, 27 wt. %, 28 wt. %, 29 wt. %, 30 wt. %, 31 wt. %, or 32 wt. % And separator gas stream 314 may comprise hydrogen in the range 20 to 28 wt. % and ranges and values there between including 20 wt. %, 21 wt. %, 22 wt. %, 23 wt. %, 24 wt. %, 25 wt. %, 26 wt. %, 27 wt. %, or 28 wt. % When the hydrogen content is at this level or more, separator gas stream 314 may be considered a hydrogen rich stream. Separator gas stream 314 flows from vessel V-304 at a temperature of approximately −35° C. or a temperature in the range −40° C. to −30° C. and ranges and values there between including −40° C., −39° C., −38° C., −37° C., −36° C., −35° C., −34° C., −33° C., −32° C., −31° C., or −30° C.

If separator gas stream contains approximately 28 wt. % propylene, for example, that could amount to approximately 10 wt. % of the amount of propylene in reactor effluent gas stream 301. Thus, to recover over 90% of the total propylene in reactor effluent gas stream 301 would require recovering at least some of the propylene from separator gas stream 314. To do so, separator gas stream 314 may be cooled in cold box H-311 to a temperature of approximately −88° C. or a temperature in the range −93° C. to −73° C. and ranges and values there between including −93° C., −92° C., −91° C., −90° C., −89° C., −88° C., −87° C., −86° C., −85° C., −84° C., −83° C., −82° C., −81° C., −80° C., −79° C., −78° C., −77° C., −76° C., −75° C., −74° C., or −73° C. This cooling partially condenses separator gas stream 314 to produce stream 317, which is partially condensed. Vessel V-311 separates stream 317 into condensed fraction 318 and gas fraction 320. Condensed fraction 318 may comprise primarily propylene and propane. In embodiments of the invention, condensed fraction 318 comprises propylene in the range 48 to 56 wt. % and ranges and values there between including 48 wt. %, 49 wt. %, 50 wt. %, 51 wt. %, 52 wt. %, 53 wt. %, 54 wt. %, 55 wt. %, or 56 wt. % In embodiments of the invention, condensed fraction 318 comprises propane in the range 28 to 36 wt. % and values there between including 28 wt. %, 29 wt. %, 30 wt. %, 31 wt. %, 32 wt. %, 33 wt. %, 34 wt. %, 35 wt. %, or 36 wt. % Condensed fraction 318 may be heated in cold box H-311 to provide cooling to cold box H-311 and to form stream 319, which is routed to de-ethanizer distillation column C-401 of de-ethanizer stage S40. Gas fraction 320 is expanded in turbo-expander X-311-I to produce cold gas 321, which is used to chill heat exchanger H-311. Cold gas 321 may be at a temperature in the range −95 to 105° C. at absolute pressure in the range 12 to 22 barₐ. Heat transfer to cold gas 321, in heat exchanger H-311, causes cold gas 321 to reheat and form stream 322. Stream 322 may be expanded in turbo-expander X-311-II to produce expanded stream 323. Expanded stream 323 is used to provide further chilling to cold box H-311. Expanded stream 323 may be at a temperature in the range −83 to −102° C. at absolute pressure in the range 2 to 10 barₐ. Heat transfer to expanded stream 323, in heat exchanger H-311, causes expanded stream 323 to reheat and form stream 324. Compressor K-311 compresses stream 324 (which is hydrogen rich) to form compressed hydrogen rich stream 325. In embodiments of the invention, compressed hydrogen rich stream 325 may include primarily hydrogen and carbon dioxide, e.g., compressed hydrogen rich stream 325 may comprise hydrogen in the range 45 to 55 wt. % and ranges and values there between including 45 wt. %, 46 wt. %, 47 wt. %, 48 wt. %, 49 wt. %, 50 wt. %, 51 wt. %, 52 wt. %, 53 wt. %, 54 wt. %, or 55 wt. % Compressed hydrogen rich stream 325 at approximately 48 wt. % hydrogen is approximately 90% vol. pure hydrogen. Compressed hydrogen rich stream 325 may comprise 25 to 35 wt. % carbon dioxide and ranges and values there between including 25 wt. %, 26 wt. %, 27 wt. %, 28 wt. %, 29 wt. %, 30 wt. %, 31 wt. %, 32 wt. %, 33 wt. %, 34 wt. %, or 35 wt. % hydrogen rich stream 325 may be at absolute pressure in a range 5 to 15 barₐ.

Work produced by turbo-expander X-311-I and turbo-expander X-311-II drives compressor K-311 to recompress stream 324 to form hydrogen rich stream 325. The two turbo-expander stages (turbo-expander X-311-I and turbo-expander X-311-II) are adapted such that their operating temperature and the operating temperature of V-311 are above −140° C. In some embodiments, the two turbo-expander stages (turbo-expander X-311-I and turbo-expander X-311-II) are adapted such that their operating temperature and the operating temperature of V-311 are above −140° C. In some embodiments, the two turbo-expander stages (turbo-expander X-311-I and turbo-expander X-311-II) are adapted such that their operating temperature and the operating temperature of V-311 are above −120° C. In some embodiments, the two turbo-expander stages (turbo-expander X-311-I and turbo-expander X-311-II) are adapted such that their operating temperature and the operating temperature of V-311 are above −100° C. At temperatures above −100° C., the propylene comprised in hydrogen rich stream 325 (and thereby recovery loss) is expected to be about 1 to 5 wt. % of hydrogen rich stream 325.

Operating turbo-expander X-311-I, turbo-expander X-311-II and vessel V-311 above −100° C. may provide advantages in relation to construction and safety. Gas streams cooled by cold boxes in ethylene plants may include oxides of nitrogen (NOₓ compounds), particularly NO₂. These NOₓ compounds have low boiling points and may pass through some separation processes with hydrogen, prior to a cryogenic process. The NOₓ compounds can react with unsaturated hydrocarbons (such as olefins) to form polymers with a gum-like appearance (“NOₓ gums”). The NOₓ gums may block valves, lines, orifices, etc., thereby posing operational and safety issues in the plant. Thus, plant design and construction may take this into account (potentially increasing capital and operating costs associated with an ethylene plant). Moreover, NOₓ gums formed under cryogenic conditions are unstable and can explode. There are reported cases of explosions in ethylene plants that have been caused by NOₓ gums. See e.g. “NOₓ IN THE CRYOGENIC HYDROGEN RECOVERY SECTION OF AN OLEFINS PRODUCTION UNIT,” W. H. Henstock, Plant/Operations Progress, Vol. 5, No. 4 October, 1986. One way of addressing the foregoing operational and safety issues presented by NOₓ gums is to ensure that feedstock to ethylene plants is free or substantially free of nitrogen and oxygen. Further, equipment such as cold boxes may be washed with solvents such as methanol to remove NOₓ gums. Embodiments of the present invention may provide additional or alternative methods of addressing the issues caused by NOₓ gums. Specifically, referring to FIG. 1, in embodiments of the invention where the two turbo-expander stages (turbo-expander X-311-I and turbo-expander X-311-II) are adapted such that their operating temperature and the operating temperature of V-311 are above −100° C., NOₓ gums may accumulate less in equipment as compared to operations at temperatures below −100° C.

In embodiments of the invention, instead of cryogenic turbo-expander-compressor separation stage S31, a pressure swing adsorption unit may be applied that separates the hydrogen from the hydrocarbons. With such a pressure swing absorption unit, however, the hydrocarbons come out at a lower pressure and would need to be recompressed.

In embodiments of the invention, de-ethanizer stage S40 removes ethane and components just as volatile as or more volatile than ethane (e.g., ethylene and methane) from propylene rich streams. Propylene rich streams include separator liquid streams 305, 308, 311 and 315 from vessels V-301, V-302, V-303, and V-304, respectively. Other propylene rich streams may include stream 319 from cold box H-311. Stream 319 may comprise propylene in the range 48 to 56 wt. % and ranges and values there between including 48 wt. %, 49 wt. %, 50 wt. %, 51 wt. %, 52 wt. %, 53 wt. %, 54 wt. %, 55 wt. %, or 56 wt. % Stream 319 may comprise propane in the range 28 to 36 wt. % and ranges and values there between including 28 wt. %, 29 wt. %, 30 wt. %, 31 wt. %, 32 wt. %, 33 wt. %, 34 wt. %, 35 wt. %, or 36 wt. % The main equipment of de-ethanizer stage S40 may include de-ethanizer distillation column C-401. In embodiments of the invention, feeds to de-ethanizer distillation column C-401 are liquid, and enter the column at a tray appropriate to their composition and temperature (although the simulation described below, Example 2, assumes that all the stream feeds to de-ethanizer distillation column C-401 are mixed to form one stream, which enters de-ethanizer distillation column C-401 at the same tray).

De-ethanizer distillation column C-401 is equipped with bottom reboiler H-401 to provide heat to the bottom of de-ethanizer distillation column C-401. Further, de-ethanizer distillation column C-401 is equipped with top condenser H-402 to remove heat at the top of de-ethanizer distillation column C-401. Top condenser H-402 is a partial condenser operated at approximately −40 to −15° C. and ranges and values there between including −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C. In embodiments of the invention, the cooling in top condenser H-402 may be achieved by propylene refrigerant (gas refrigerant 532). In embodiments of the invention, top condenser H-402 may be operated in the range −40 to −20° C. such that de-ethanizer distillation column C-401 can operate at a lower temperature, which may be advantageous. Bottom reboiler H-401 may use heat from a hot water cycle (e.g. hot water originating from condensing the water in treated effluent gas stream 303 by, for example, a quench tower that could be added to the design to improve the energy efficiency), hence bottom reboiler H-401 may have a hot water circulation supply (HWCS) and a hot water circulation return (HWCR).

Distillate from the top of de-ethanizer distillation column C-401 is cooled in top condenser H-402 and separated in separation vessel V-401 to form stream 402, which may comprise C₁ to C₂ hydrocarbons (ethane, ethylene and methane). Stream 402 may be routed to an internal fuel gas network (IFGN).

Liquid product stream 403 flowing from the bottom of de-ethanizer distillation column C-401 may include propylene and propane as its primary components. In embodiments of the invention, product stream 403 may comprise propylene in the range 40 to 70 wt. % and ranges and values there between including 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. %, 61 wt. %, 62 wt. %, 63 wt. %, 64 wt. %, 65 wt. %, 66 wt. %, 67 wt. %, 68 wt. %, 69 wt. %, or 70 wt. %. In embodiments of the invention, liquid product stream 403 may comprise propane in the range 30 to 60 wt. % and ranges and values there between including 30 wt. %, 31 wt. %, 32 wt. %, 33 wt. %, 34 wt. %, 35 wt. % 36 wt. %, 37 wt. %, 38 wt. %, 39 wt. %, 40 wt. % 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, or 60 wt. %. In embodiments of the invention the amount of propylene in product stream 403 may comprise most of the propylene entering system 10, in reactor effluent gas stream 301. For example, 90 wt. % or more of propylene in reactor effluent gas stream 301 may be recovered in product stream 403. In embodiments of the invention 97 wt. % or more of propylene in reactor effluent gas stream 301 may be recovered in product stream 403. In embodiments of the invention 99 wt. % or more of propylene in reactor effluent gas stream 301 may be recovered in product stream 403.

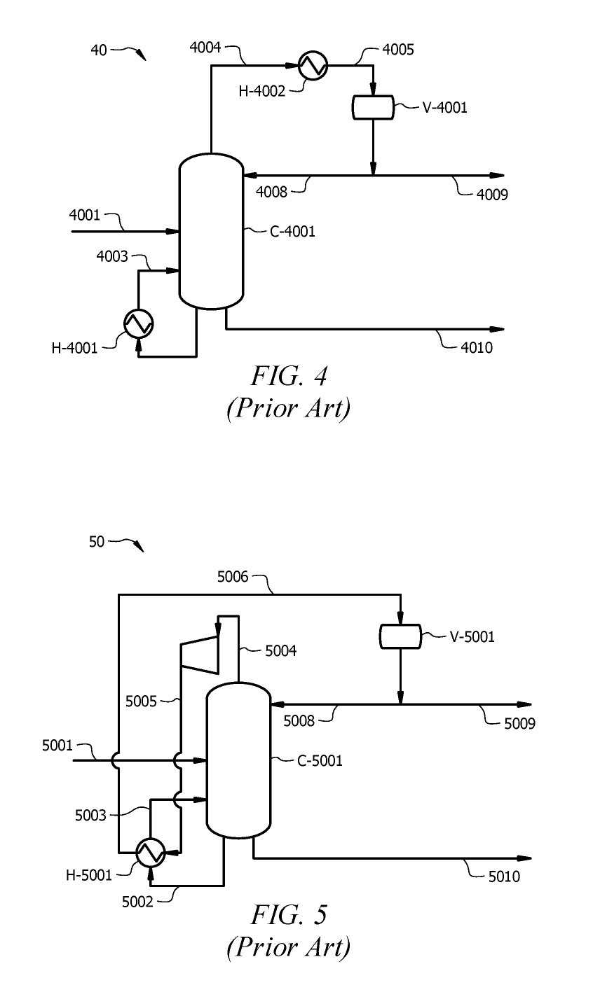

Product stream 403 may need further processing to meet product specifications for polymer grade propylene. This may be done in a C₃-splitter column (e.g., as shown in FIG. 4 and FIG. 5). Examples 3 and 4 below show how systems 40 and 50 of FIGS. 4 and 5, respectively, may be used to produce polymer grade propylene.

In embodiments of the invention, propylene refrigeration cycle S50 may include the use of four stage propylene compressor K-501 (including K-501-1, K-501-11, K-501-111, and K-501-1V). The pressurized propylene gas may be condensed against cooling water in heat exchanger H-501, hence heat exchanger H-501 having cooling water supply (CWS) and cooling water return (CWR) shown in FIG. 1. Vessels V-5-1, V-502, V-503, V-504 may receive at a portion cooled refrigerant 501 as liquid refrigerants 513, 523, and 526. Vessels V-5-1, V-502, V-503, V-504, and V-505 separates liquid propylene refrigerant from vapor propylene refrigerant and provides a feed of liquid refrigerants 511, 521, 526, and 523, which are used to cool treated effluent gas stream 303 and portions thereof in S30 precool train stage. Liquid refrigerants 511, 521, 526, and 533 are heated and vaporized in H-301, H-302, H-303, and H-304 to form gas refrigerants 512, 522, 527, and 534. Liquid refrigerant 531 is used for cooling in top condenser H-402, where it is reheated to form gas refrigerant 532. In embodiments of the invention, an ethylene refrigeration compressor (typically used to provide cooling in the −40° C. to 100° C. temperature range) is not included in the system. From the heat exchangers, gas refrigerants 512, 522, 527, 534 and 532 are routed to Vessels V-5-1, V-502, V-503, V-504, and V-505, which in turn supplies streams 502, 503, 504, 505, 506, and 509 for compression in compressor K-501.

In embodiments of the invention, treated effluent gas stream 303 may be compressed to higher pressures, so that the temperatures to achieve sufficient propylene recovery can be raised, and only one turbo expander may be used, in cryogenic turbo-expander-compressor separation stage S31, instead of two turbo expanders. In embodiments of the invention, treated effluent gas stream 303 may be compressed to 15-40 barₐ and ranges and values there between including 15 barₐ, 16 barₐ, 17 barₐ, 18 barₐ, 19 barₐ, 20 barₐ, 21 barₐ, 22 barₐ, 23 barₐ, 24 barₐ, 25 barₐ, 26 barₐ, 27 barₐ, 28 barₐ, 29 barₐ, 30 barₐ, 31 barₐ, 32 barₐ, 33 barₐ, 34 barₐ, 35 barₐ, 36 barₐ, 37 barₐ, 38 barₐ, 39 barₐ, or 40 barₐ. An advantage of lower pressures (e.g., 15-25 barₐ) is that treated effluent gas stream 303 may need less compressor power and the equipment is at lower pressure. A disadvantage of lower pressures (e.g., 15-25 barₐ) is that cooling to lower temperatures may be required and that cooling duty may increase. An advantage of higher pressures (e.g., 25-40 barₐ) is that temperatures may be higher and cooling duties may reduce, but at the cost higher pressure equipment and more compressor power for the reactor gas. Thus, embodiments of the invention may configured taking these advantages and disadvantages into account.

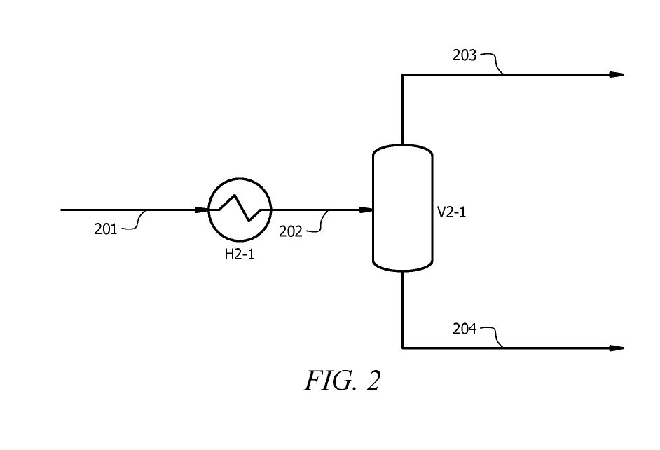

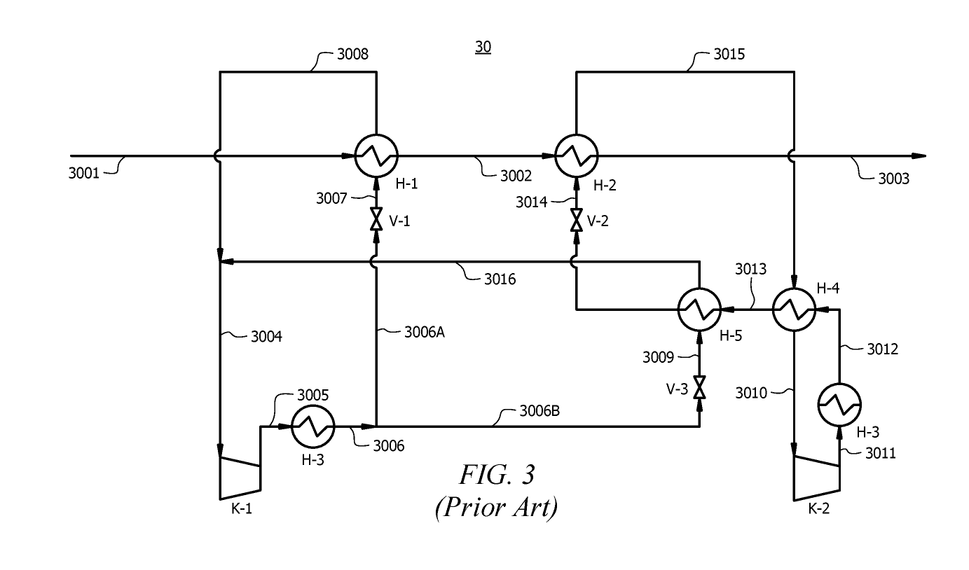

The following prophetic simulation examples based on a simplified product cooling and separation system, shown in FIG. 2, illustrate the problem in recovering propylene.

### EXAMPLES

### Example 1

### Prophetic Simulated Example

The prophetic simulated example discussed herein in relation to FIG. 2 and

Tables 1 and 2 are based on calculations made with Aspen Plus® modelling software. The simulation is based on a propane or propylene compressor refrigeration cycle that is capable of cooling to temperatures of about −40° C. The simulated process also includes a distillation column with a partial condenser operating at 22 barₐ and −35° C. and a reboiler operated at approximately 60° C. The distillation column can be cooled with a compressor refrigeration system and is able to separate C₂₋ components from C₃₊ components. The prophetic simulation example assumes reactor effluent stream 201 flowing at a rate of 100 tonne/hour (t/h). Reactor effluent 201 is a mixture of 5 wt. % hydrogen and 95 wt. % propylene at absolute pressure of 25 barₙ and temperature of 30° C. In the simulation, reactor effluent stream 201 is cooled in heat exchanger H2-1 to a temperature of −35° C. to form stream 202. Further, distillation tower V2-1 separates stream 202 into vapor fraction 203 and liquid fraction 204. The mass flow of vapor fraction 203 is 14.2 t/h, of which 9.3 t/h is propylene. Table 1 shows stream properties calculated from the simulation, if heat exchanger H2-1 cools reactor effluent stream 201 so that stream 202 is at a temperature of −35° C.

TABLE 1
Stream table for reactor effluent cooling to −35° C.

| | | 201 | 202 | 203 | 204 |
|---|---|---|---|---|---|
| Pressure | barₐ | 25 | 25 | 25 | 25 |
| Temperature | ° C. | 30 | −35 | −35 | −35 |
| Mass Flow | t/h | 100 | 100 | 14.2 | 85.8 |
| Hydrogen mass flow | t/h | 5 | 5 | 4.9 | 0.1 |
| Propylene mass flow | t/h | 95 | 95 | 9.3 | 85.7 |

Table 2 shows stream properties calculated if heat exchanger H2-1 cools reactor effluent stream 201 so that stream 202 is at a temperature of −90° C.

TABLE 2
Stream table for effluent cooling to −90° C.

| | Units | 201 | 202 | 203 | 204 |
|---|---|---|---|---|---|
| Pressure | barₐ | 25 | 25 | 25 | 25 |
| Temperature | ° C. | 30 | −90 | −90 | −90 |
| Mass Flow | t/h | 100 | 100 | 5.5 | 94.5 |
| Hydrogen mass flow | t/h | 5 | 5 | 5.0 | 0.0 |
| Propylene mass flow | t/h | 95 | 95 | 0.5 | 94.5 |

As Table 1 and Table 2 show, the lower the temperature to which the reactor effluent stream is cooled, the higher the recovery of propylene.

### Example 2

### Prophetic Simulated Example

A simulation of an embodiment of system 10 was performed with Aspen Plus® 8.2 process simulation software. It should be noted that, in the simulation, all streams entering de-ethanizer distillation column C-401 were assumed to be mixed to form one stream and that stream was assumed to enter de-ethanizer distillation column C-401 at the same tray. Table 3 and Table 4 show the heat and mass balances and material balances, respectively, based on this simulation. The following assumptions were made in the simulation:
1. Steam and hot water are assumed to be generated with 90% thermal efficiency (LHV).
2. Electricity is assumed to be generated with 50% thermal efficiency (LHV).
3. Electric motors have an efficiency of 95%
4. Compressors and expanders have an isentropic efficiency of 75%

Based on the simulation, propylene recovery using the separation process described is the propylene present in stream 403, which is 75.1 t/h. This would be a propylene recovery of 99.4%. It should be noted that embodiments of the invention may be implemented such that the content and properties of the streams shown in Table 3 and Table 4 is different from that disclosed in the tables. For example, the values in Table 3 and Table 4 may, in embodiments of the invention, fall within a range of plus or minus 20% of the value shown.

TABLE 3

| STREAM | NO. | 303 | 304 | 305 | 306 | 307 | 308 | 309 | 310 |
|---|---|---|---|---|---|---|---|---|---|
| PRESSURE | BARA | 25.0 | 25.0 | 0.0 | 25.0 | 25.0 | 25.0 | 25.0 | 25.0 |
| TEMPERATURE | ° C. | 50 | 30 | 0 | 30 | 10 | 10 | 10 | −15 |
| MASS FLOW | t/h | 156 | 156 | 0 | 156 | 156 | 63 | 93 | 93 |
| VOLUME FLOW | m3/s | 1.88 | 1.73 | 0.00 | 1.73 | 1.34 | 0.03 | 1.30 | 0.98 |
| PHASE | V/L | V | V | L | V | V/L | L | V | V/L |
| STREAM | NO | 501 | 502 | 503 | 504 | 505 | 506 | 507 | 508 |
| PRESSURE | BARA | 1.6 | 3.0 | 3.0 | 7.0 | 7.0 | 12.0 | 12.0 | 12.0 |
| TEMPERATURE | ° C. | −39 | −5 | −12 | 32 | 22 | 62 | 44 | 44 |
| MASS FLOW | t/h | 96 | 96 | 184 | 184 | 302 | 302 | 427 | 427 |
| VOLUME FLOW | m3/s | 7.85 | 4.41 | 8.19 | 3.94 | 6.19 | 3.81 | 5.18 | 5.18 |
| PHASE | V/L | V | V | V | V | V | V | V | V |
| STREAM | NO. | 311 | 312 | 313 | 314 | 315 | 317 | 318 | 319 |
| PRESSURE | BARA | 25.0 | 25.0 | 25.0 | 25.0 | 25.0 | 25.0 | 25.0 | 25.0 |
| TEMPERATURE | ° C. | −15 | −15 | −35 | −35 | −35 | −88 | −88 | −40 |
| MASS FLOW | t/h | 51 | 42 | 42 | 26 | 16 | 26 | 13 | 13 |
| VOLUME FLOW | m3/s | 0.03 | 0.95 | 0.81 | 0.80 | 0.01 | 0.58 | 0.01 | 0.01 |
| PHASE | V/L | L | V | V/L | V | L | V/L | L | L |
| STREAM | NO | 509 | 510 | 511 | 512 | 513 | 521 | 522 | 523 |
| PRESSURE | BARA | 22.0 | 22.0 | 22.0 | 12.0 | 22.0 | 12.0 | 7.0 | 12.0 |
| TEMPERATURE | ° C. | 81 | 53 | 53 | 27 | 53 | 27 | 6 | 27 |
| MASS FLOW | t/h | 427 | 427 | 29 | 29 | 398 | 88 | 88 | 215 |
| VOLUME FLOW | m3/s | 2.91 | 0.26 | 0.02 | 0.32 | 0.24 | 0.05 | 1.66 | 0.12 |
| PHASE | V/L | V | L | L | V | L | L | V | L |
| STREAM | NO. | 320 | 321 | 322 | 323 | 324 | 325 | 402 | 403 |
| PRESSURE | BARA | 25.0 | 18.0 | 18.0 | 6.0 | 6.0 | 10.4 | 22.0 | 23.0 |
| TEMPERATURE | ° C. | −88 | −99 | −40 | −88 | −40 | 17 | −21 | 58 |
| MASS FLOW | t/h | 13 | 13 | 13 | 13 | 13 | 13 | 6 | 136 |
| VOLUME FLOW | m3/s | 0.57 | 0.74 | 1.00 | 2.36 | 2.98 | 2.15 | 0.06 | 0.09 |
| PHASE | V/L | V | V | V | V | V | V | V | L |
| STREAM | NO. | 526 | 527 | 528 | 530 | 531 | 532 | 533 | 534 |
| PRESSURE | BARA | 7.0 | 3.0 | 7.0 | 3.0 | 3.0 | 1.5 | 3.0 | 1.5 |
| TEMPERATURE | ° C. | 6 | −21 | −6 | −21 | −21 | −39 | −21 | −39 |
| MASS FLOW | t/h | 70 | 70 | 113 | 96 | 72 | 72 | 24 | 24 |
| VOLUME FLOW | m3/s | 0.04 | 3.02 | 0.06 | 0.05 | 0.03 | 5.90 | 0.01 | 1.95 |
| PHASE | V/L | L | V | L | L | L | V | L | V |

TABLE 4

| STREAM | NO. | 303 | 304 | 305 | 306 | 307 | 308 |
|---|---|---|---|---|---|---|---|
| HYDROGEN | t/h | 6.3 | 6.3 | 0.0 | 6.3 | 6.3 | 0.0 |
| METHANE | t/h | 1.0 | 1.0 | 0.0 | 1.0 | 1.0 | 0.0 |
| ETHYLENE | t/h | 1.8 | 1.8 | 0.0 | 1.8 | 1.8 | 0.3 |
| ETHANE | t/h | 5.6 | 5.6 | 0.0 | 5.6 | 5.6 | 1.1 |
| PROPYLENE | t/h | 75.5 | 75.5 | 0.0 | 75.5 | 75.5 | 32.4 |
| PROPANE | t/h | 61.5 | 61.5 | 0.0 | 61.5 | 61.5 | 28.8 |
| N-BUTANE | t/h | 0.01 | 0.01 | 0.00 | 0.01 | 0.01 | 0.01 |
| 1-BUTENE | t/h | 0.0 | 0.0 | 0.0 | 0.0 | 0.0 | 0.0 |
| BENZENE | t/h | 0.0 | 0.0 | 0.0 | 0.0 | 0.0 | 0.0 |
| NITROGEN | t/h | 0.0 | 0.0 | 0.0 | 0.0 | 0.0 | 0.0 |
| OCYGEN | t/h | 0.0 | 0.0 | 0.0 | 0.0 | 0.0 | 0.0 |
| CARBON DIOXIDE | t/h | 0.0 | 0.0 | 0.0 | 0.0 | 0.0 | 0.0 |
| WATER | t/h | 0.0 | 0.0 | 0.0 | 0.0 | 0.0 | 0.0 |
| CARBON MONXIDE | t/h | 3.9 | 3.9 | 0.0 | 3.9 | 3.9 | 0.07 |
| TOTAL | t/h | 155.5 | 155.5 | 0.0 | 155.5 | 155.5 | 62.8 |
| STREAM | NO. | 309 | 310 | 311 | 312 | 313 | 314 |
| HYDROGEN | t/h | 6.2 | 6.2 | 0.0 | 6.2 | 6.2 | 6.2 |
| METHANE | t/h | 1.0 | 1.0 | 0.1 | 0.9 | 0.9 | 0.9 |
| ETHYLENE | t/h | 1.5 | 1.5 | 0.3 | 1.1 | 1.1 | 1.0 |
| ETHANE | t/h | 4.4 | 4.4 | 1.4 | 3.0 | 3.0 | 2.4 |
| PROPYLENE | t/h | 43.1 | 43.1 | 27.0 | 16.1 | 16.1 | 7.2 |
| PROPANE | t/h | 32.6 | 32.6 | 21.9 | 10.8 | 10.8 | 4.3 |
| N-BUTANE | t/h | 0.00 | 0.00 | 0.00 | 0.00 | 0.00 | 0.00 |
| 1-BUTENE | t/h | 0.0 | 0.0 | 0.0 | 0.0 | 0.0 | 0.0 |
| BENZENE | t/h | 0.0 | 0.0 | 0.0 | 0.0 | 0.0 | 0.0 |
| NITROGEN | t/h | 0.0 | 0.0 | 0.0 | 0.0 | 0.0 | 0.0 |
| OCYGEN | t/h | 0.0 | 0.0 | 0.0 | 0.0 | 0.0 | 0.0 |
| CARBON DIOXIDE | t/h | 0.0 | 0.0 | 0.0 | 0.0 | 0.0 | 0.0 |
| WATER | t/h | 0.0 | 0.0 | 0.0 | 0.0 | 0.0 | 0.0 |
| CARBON MONXIDE | t/h | 3.8 | 3.8 | 0.07 | 3.7 | 3.7 | 3.7 |
| TOTAL | t/h | 92.7 | 92.7 | 50.7 | 41.9 | 41.9 | 25.7 |
| STREAM | NO. | 315 | 317 | 318 | 319 | 320 | 321 |
| HYDROGEN | t/h | 0.0 | 6.2 | 0.0 | 0.0 | 6.2 | 6.2 |
| METHANE | t/h | 0.0 | 0.9 | 0.0 | 0.0 | 0.8 | 0.8 |
| ETHYLENE | t/h | 0.2 | 1.0 | 0.5 | 0.5 | 0.5 | 0.5 |
| ETHANE | t/h | 0.6 | 2.4 | 1.5 | 1.5 | 0.8 | 0.8 |
| PROPYLENE | t/h | 8.9 | 7.2 | 6.8 | 6.8 | 0.4 | 0.4 |
| PROPANE | t/h | 6.5 | 4.3 | 4.1 | 4.1 | 0.2 | 0.2 |
| N-BUTANE | t/h | 0.00 | 0.00 | 0.00 | 0.00 | 0.00 | 0.00 |
| 1-BUTENE | t/h | 0.0 | 0.0 | 0.0 | 0.0 | 0.0 | 0.0 |
| BENZENE | t/h | 0.0 | 0.0 | 0.0 | 0.0 | 0.0 | 0.0 |
| NITROGEN | t/h | 0.0 | 0.0 | 0.0 | 0.0 | 0.0 | 0.0 |
| OCYGEN | t/h | 0.0 | 0.0 | 0.0 | 0.0 | 0.0 | 0.0 |
| CARBON DIOXIDE | t/h | 0.0 | 0.0 | 0.0 | 0.0 | 0.0 | 0.0 |
| WATER | t/h | 0.0 | 0.0 | 0.0 | 0.0 | 0.0 | 0.0 |
| CARBON MONXIDE | t/h | 0.0 | 3.7 | 0.0 | 0.0 | 3.7 | 3.7 |
| TOTAL | t/h | 16.2 | 25.7 | 13.0 | 13.0 | 12.7 | 12.7 |
| STREAM | NO. | 322 | 323 | 324 | 325 | 402 | 403 |
| HYDROGEN | t/h | 6.2 | 6.2 | 6.2 | 6.2 | 0.09 | 0.00 |
| METHANE | t/h | 0.8 | 0.8 | 0.8 | 0.84 | 0.16 | 0.00 |
| ETHYLENE | t/h | 0.5 | 0.5 | 0.5 | 0.52 | 1.2 | 0.00 |
| ETHANE | t/h | 0.8 | 0.8 | 0.8 | 0.85 | 4.7 | 0.00 |
| PROPYLENE | t/h | 0.4 | 0.4 | 0.4 | 0.42 | 0.04 | 75.1 |
| PROPANE | t/h | 0.2 | 0.2 | 0.2 | 0.19 | 0.00 | 61.3 |
| N-BUTANE | t/h | 0.00 | 0.00 | 0.00 | 0.00 | 0.00 | 0.01 |
| 1-BUTENE | t/h | 0.0 | 0.0 | 0.0 | 0.0 | 0.0 | 0.0 |
| BENZENE | t/h | 0.0 | 0.0 | 0.0 | 0.0 | 0.0 | 0.0 |
| NITROGEN | t/h | 0.0 | 0.0 | 0.0 | 0.0 | 0.0 | 0.0 |
| OCYGEN | t/h | 0.0 | 0.0 | 0.0 | 0.0 | 0.0 | 0.0 |
| CARBON DIOXIDE | t/h | 0.0 | 0.0 | 0.0 | 0.0 | 0.0 | 0.0 |
| WATER | t/h | 0.0 | 0.0 | 0.0 | 0.0 | 0.0 | 0.0 |
| CARBON MONXIDE | t/h | 3.7 | 3.7 | 3.7 | 3.7 | 0.20 | 0.00 |
| TOTAL | t/h | 12.7 | 12.7 | 12.7 | 12.7 | 6.4 | 136.4 |

### Example 3

### Prophetic Simulated Example

Referring to FIG. 4, shown is system 40, a prior art system, for purifying a C₃ stream from a steam cracker to form polymer-grade propylene by fractionating with heat input from quench water and cooled against cooling water. The propane may be recycled back to the reactor.

In system 40, 20 t/h of stream 4001 (liquid C₃ product) contains 5 wt. % propane, 5 wt. % propylene and is fed to stage 78 of distillation column C-4001 (which has 160 stages and an internal diameter of 4 meters). The pressure drop over distillation column C-4001 is 1.3 barₐ. Reboiler H-4001 has a duty of 18.8 MWth and produces stream 4003, which is a flow of 235 t/h of vapor. Distillation column C-4001 produces 215 t/h of vapor at the top, stream 4004, which is condensed against cooling water in heat exchanger H-4002 to form stream 4005. Stream 4005 is sent to vessel V-4001, where 196 t/h is pumped back as reflux stream 4008 and 19 t/h of 99% pure propylene is produced as stream 4009. Stream 4010 includes propane. The condenser operates at a pressure of 16 barₐ, which allows the heat from condenser H-4002 to be rejected to colder cooling water. Distillation column C-4001 is operated at a vapor velocity at 79% of the flooding velocity.

An advantage of system 40 is that it can use low value waste heat (quenchwater) from the steam cracking process as heat input. A disadvantage of system 40 is that it may have to operate at high pressure (making it capital intensive) and the higher pressure makes the distillation harder, requiring more reflux, which may cause an increase in column diameter.

### Example 4

### Prophetic Simulated Example

Referring to FIG. 5, shown is a prior art system, system 50, for purifying a C₃ stream from a steam cracker to form polymer-grade propylene by fractionating with vapor recompression system. In system 50, 25.3 t/h of liquid C₃ product, stream 5001 comprising 5 wt. % propane, 5 wt. % propylene is fed to stage 78 of distillation column C-201 (which has 160 stages). The pressure drop over the column is 1.3 barₐ. Distillation column C-201 produces 214 t/h of vapor at the top, stream 5004, which is compressed to about 14 barₐ to form stream 5005. Stream 5005 may be condensed to stream 5006 in heat exchanger H-5001, where the heat is rejected to bottom product 5002 of distillation column C-201. Bottom product 5002 boils to form stream 5003. Condensed liquid 5006 is fed through vessel V-5001 back as reflux 5008 (214 t/h) and as stream 5009, a 99 wt. % pure propylene product. Stream 5010 includes propane.

An advantage of system 50 is that it operates at a lower pressure (9 bara) and that the distillation is easier, requiring less trays and or less reflux, resulting in a cheaper column design. A disadvantage of system 50 is that it may require a compressor to work and the compressor requires high value energy, such as electricity (motor drive) or high pressure steam (steam turbine drive) to function.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

Moreover, the scope of the present application is not intended to be limited to the particular embodiments of the process, machine, manufacture, composition of matter, means, methods and steps described in the specification.

1.-20. (canceled)

21. A separation process to recover propylene from effluent of a propane dehydrogenation reactor, the process comprising:
cooling the effluent to produce a gas stream, wherein hydrogen and propylene collectively comprises the major component of the gas stream;
cooling the gas stream in a cooling unit that comprises one or more turbo-expanders, wherein the one or more turbo-expanders do not cool any portion of the gas stream below −140° C.;
flowing condensate from the cooling unit to a de-ethanizer unit, the de-ethanizer unit adapted to remove ethane; and
flowing, from the de-ethanizer unit, a liquid stream comprising propylene.

22. The process of claim 21, wherein the one or more turbo-expanders do not cool any portion of the gas stream below −120° C.

23. The process of claim 21, wherein the one or more turbo-expanders do not cool any portion of the gas stream below −100° C.

24. The process of claim 21, wherein the cooling unit further comprises a cold box and a separation vessel.

25. The process of claim 24, wherein the gas stream is cooled to a temperature within a range of −25° C. to −45° C. and at least a portion of the gas stream is subsequently cooled by the cold box to a temperature within a range of −78° C. to −98° C.

26. The process of claim 24, wherein the cold box cools the gas stream so that a portion of the gas stream condenses, thereby forming a stream including cold box condensate and cold box vapor.

27. The process of claim 24, further comprising:
flowing the stream including cold box condensate and cold box vapor to the separation vessel;
separating, by the separation vessel, the stream including cold box condensate and cold box vapor into a separate stream of cold box condensate and a separate stream of cold box vapor;
expanding the separate stream of cold box vapor in the one or more turbo-expanders;
flowing the expanded cold box vapor from the one or more turbo-expanders to the cold box to cool the cold box and thereby produce a reheated cold box stream; and
expanding the reheated cold box stream in the one or more turbo-expanders.

28. The process of any of claim 21, wherein the de-ethanizer unit comprises a distillation column.

29. The process of claim 21, wherein the cooling of the effluent to produce the gas stream comprises heat transfer and separation processes carried out in a series of units, wherein each unit comprises a heat exchanger that cools influent of the heat exchanger and a vessel that separates effluent of the heat exchanger into vapor and condensate.

30. The process of claim 21, wherein 90 wt. % or more of propylene present in the effluent of the propane dehydrogenation reactor is recovered in the liquid stream comprising propylene.

31. The process of claim 21, wherein 97 wt. % or more of propylene present in the effluent of the propane dehydrogenation reactor is recovered in the liquid stream comprising propylene.

32. The process of claim 21, wherein 99 wt. % or more of propylene present in the effluent of the propane dehydrogenation reactor is recovered in the liquid stream comprising propylene.

33. The process of claim 21, wherein the liquid stream further comprises propane.

34. The process of claim 21, wherein the effluent of the propane dehydrogenation reactor comprises mainly propylene and propane.

35. The process of claim 32, wherein the effluent of the propane dehydrogenation reactor further comprises water (H₂O), carbon dioxide (CO₂), hydrogen, ethane, methane, ethylene.

36. The process of claim 21, further comprising:
removing water and carbon dioxide (CO₂) from the effluent of the propane dehydrogenation reactor prior to cooling.

37. The process of claim 21, wherein the effluent of the propane dehydrogenation reactor is compressed prior to cooling.

38. The process of claim 21, wherein the cooling unit has only one turbo-expander.

39. The process of claim 21, wherein the de-ethanizer unit is adapted to remove ethane, or methane, or ethylene, or combinations thereof from the condensate from the cooling unit.

40. The process of claim 21, wherein a portion of the gas stream is recovered as 45 to 55 wt. % of hydrogen.
